(19) United States
(12) Patent Application Publication
(10) Pub. No.: US 2009/0299330 A1
(43) Pub. Date: Dec. 3, 2009

(54) Cellular Delivery Device

(76) Inventors: Robroy H. Mac Iver, Seattle, WA (US); Daniel A. Harrington, Houston, TX (US); Constantino Mavroudls, Cleveland, OH (US); Carl L. Backer, Chicago, IL (US); Robert D. Stewart, Chapel Hill, NC (US)

Correspondence Address: THE LAW OFFICE OF EDWARD L BISHOP, 1240 S. OLD FORGE CT., PALATINE, IL, 60067, US

(21) Appl. No.: 12/431,185

(22) Filed: Apr. 28, 2009

Related U.S. Application Data
(60) Provisional application No. 61/048,391, filed on Apr. 28, 2008.

(51) Int. Cl. A61M 5/00

(52) U.S. Cl. 604/522; 604/57

(57) ABSTRACT

A cellular delivery device comprising a cellular carrier and an insertion device is provided. The cellular carrier includes at least one elongate strand of biocompatible material with a distal end and a proximal end. A stop member is connected to the proximal end of the elongate strand. The insertion device includes a needle attached to a hollow tubular body. The hollow tubular body is sized to receive the cellular carrier such that at least a portion of the elongate strand may be located within the hollow tubular body and the stop member protrudes from the proximal end of the hollow tubular body.

### CROSS REFERENCE TO RELATED APPLICATION

This application claims priority to U.S. Provisional Patent Application Ser. No. 61/048,391, filed on Apr. 28, 2008, and incorporated herein by reference.

### TECHNICAL FIELD OF THE INVENTION

The invention relates generally to medical devices and more particularly to a device for inserting cellular material into living tissue.

### BACKGROUND OF THE INVENTION

The insertion of cellular material into living tissue may aid in replacing native tissue that is damaged or destroyed or facilitate the regeneration of native tissue. It may be useful to introduce cellular material in a variety of tissues throughout the body of a living organism, e.g., nerves of the spinal cord, severed muscles, or the heart. One example of where it may be useful to introduce cellular material into living tissue is the human heart.

The human heart contains four chambers: the right atrium, the right ventricle, the left atrium, and the left ventricle. Within the lower left portion of a person's right atrium is specialized tissue that conducts electrical signals from the atria to the ventricles. This specialized tissue is called the atrioventricular node (AV node). The failure of the AV node to work normally may cause abnormally slow heart rates (bradycardia, bradyarrythmia) or an abnormal heart rhythm.

AV node failure is often categorized as either Type I, Type II, or Type III heart block. Type I heart block is characterized by a slowing of the electrical signals from the sinoatrial node to the AV node. This is the least severe form of heart block and may produce no discernable symptoms. Type II heart block is more severe; it involves the failure of some electrical signals from the atria to reach the ventricles. Persons suffering from Type II heart block may experience a slowing of the heart rate, resulting in fatigue, dizziness, shortness of breath, or fainting. In Type III heart block there is no conduction through the AV node. Thus, no electrical signals from the atria reach the ventricles. Type III heart block may be life threatening.

There are different etiologies for the failure of the AV node. The complete failure of the AV node, or Type III heart block, happens “spontaneously” in 1 of every 14,000 to 20,000 births. The cause of this “spontaneous” failure is usually antibodies developed by the mother that attack the fetus.

AV node failure may also occur during congenital heart surgery. AV node failure occurs in about 2-3% of such operations on the heart. The AV node is located close to several valves of the heart, including the tricuspid valve, which connects the right atrium and the right ventricle. During surgery to repair the valves, there is a risk of damaging the AV node. AV node failure is particularly prevalent during surgery on the hearts of infants and children because the small size of their hearts makes it difficult to perform surgery on one portion of the heart without affecting the other parts of the heart.

Artificial pacemakers are currently used to treat AV node failure. However, it may be advantageous to regrow the AV node rather than replacing its function with an artificial pacemaker. This is particularly true for children with AV node problems because they will likely need pacemaker repair or replacement over the course of their lives.

One previous technique involves attaching a construct containing cellular tissue to the outer surface of the heart tissue. Because the heart is a mass of muscle and is constantly moving, it may be difficult to attach a construct to the surface of the tissue such that the cells successfully grow. In addition, when placed on the surface of the heart tissue, the cells may be distanced from the target-tissue's blood supply, making them less likely to grow.

A second technique involves injecting cells in a fluid medium into the heart tissue. This technique has not been highly successful because it is difficult to control the orientation of the cells during the injection process. The cells often do not take hold and thrive after injection.

### SUMMARY OF THE INVENTION

A cellular delivery device having a cellular carrier and an insertion device is described. The cellular carrier has at least one elongate strand of biocompatible or biodegradable material. The elongate strand of the cellular carrier has a distal and a proximal end. A stop member is connected to the proximal end of the elongate strand. The insertion device includes a needle attached to a hollow tubular body. The hollow tubular body is sized to receive the cellular carrier such that at least a portion of the elongate strand may be located within the hollow tubular body and the stop member protrudes from the proximal end of the hollow tubular body.

The cellular delivery device may be used to deliver cellular material to living tissue by placing or growing cellular material on said cellular carrier. The cellular carrier may be received into a cavity in the proximal end of the insertion device such that the stop member protrudes from the proximal end of the insertion device. A first point on the surface of living tissue may be penetrated by the needle of the insertion device. The insertion device may be passed through the tissue and out of the tissue through a second point in the surface of the tissue. As the insertion device is passed through the tissue, the stop member may be allowed to catch on the surface of the tissue thereby preventing the cellular carrier from moving any further through the tissue. Thus, after the insertion device is removed, the cellular carrier may be left embedded within the tissue.

Other embodiments, systems, methods, features, and advantages of the present invention will be, or will become, apparent to one having ordinary skill in the art upon examination of the following drawings and detailed description. It is intended that all such additional systems, method, features, and advantages be within the scope of the present invention, and can be protected by the accompanying claims.

### BRIEF DESCRIPTION OF THE DRAWINGS

The invention may be better understood with reference to the following drawings. The components in the drawings are not necessarily to scale, emphasis instead being placed upon illustrating the principles of the invention. In the drawings, like reference numbers designate corresponding parts throughout.

FIG. 1 is a perspective view of a cellular carrier device;

FIG. 2 is a perspective view of an insertion device;

FIG. 3 is a side-sectional view of the insertion device of FIG. 2;

FIG. 4 is a side-sectional view of a cellular delivery device including the cellular carrier of FIG. 1 and the insertion device of FIG. 2;

FIG. 5 is a side-sectional view of a cellular delivery device;

FIG. 6 is a perspective view of a cellular carrier device;

FIG. 7 is a perspective view of a cellular carrier device;

FIG. 8 is a perspective view of a cellular delivery device including the cellular carrier of FIG. 7;

FIG. 9 is a side-sectional view of the cellular delivery device of FIG. 8;

FIG. 10 is a cross-sectional view of a human heart showing the insertion of a cellular delivery device;

FIG. 11 is a partial cross-sectional view of the human heart shown in FIG. 10 showing the insertion of a cellular delivery device;

FIG. 12 is a partial cross-sectional view of the human heart shown in FIG. 10, showing the withdrawal of the insertion device;

FIG. 13 is a partial cross-sectional view of the human heart shown in FIG. 10, showing the cellular carrier embedded in tissue.

### DETAILED DESCRIPTION OF THE DRAWINGS AND THE PRESENTLY

### Preferred Embodiments

The following descriptions of detailed embodiments are for exemplifying the principles and advantages of the inventions. They are not to be taken in any way as limitations on the scope of the inventions.

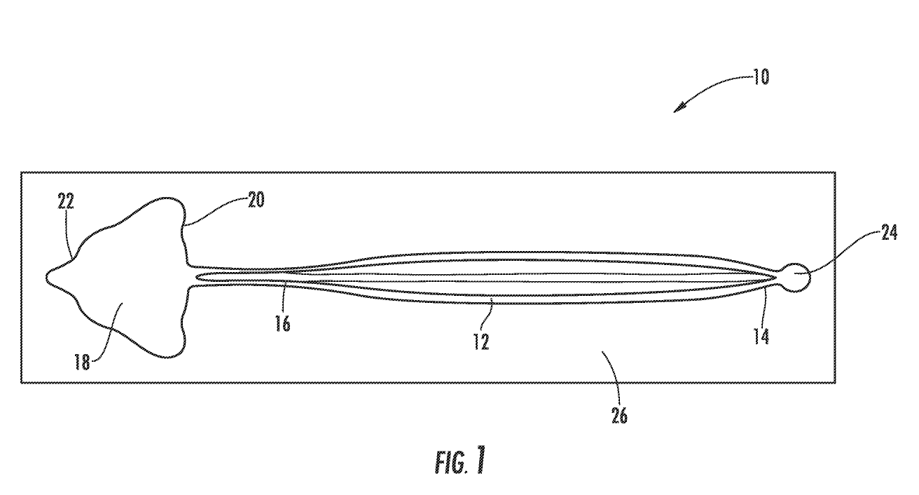

Referring now to the drawings, particularly FIG. 1, a cellular carrier 10 is shown. In the present application, the term “proximal” refers to the direction that is generally toward a physician during a medical procedure, while the term “distal” refers to a direction that is generally towards a target site within a patient's anatomy during a medical procedure.

The cellular carrier shown in FIG. 1 has three elongate strands 12, each with a distal end 14 and a proximal end 16. A distal member 24 is coupled to the distal end 14 of the elongate strand 12. A stop member 18, having a distal end 20 and a proximal end 22, is connected to the proximal ends 16 of the elongate strands 12. The elongate strands 12 and stop member 18 together form a cellular carrier 10 that is capable of carrying cellular material as the elongate strands 16 are moved through living tissue, as explained in further detail below.

FIG. 1 shows the cellular carrier 10 resting in a half tube 26. Placing the cellular carrier 10 with the half tube 26 may facilitate growing cells on the cellular carrier 10. The half tube 26 may be formed by splitting a tube in half and placing the half tube concave-side up in a nutrient rich medium suitable for growing cells. The cellular carrier 10 is then placed inside the half tube 26. As the cells grow on the cellular carrier 10, they may stretch and stick to the carrier 10. This forms a cellular construct that is secured to the cellular carrier 10. The half tube 26 may be made of any suitable material known in the art, such as silicone, PFTE, metal, plastic, or any other suitable material. For embodiments which have cells grown on the carrier, any other device or method known in the art for growing cells may be used instead of a half tube.

FIG. 1 shows an embodiment having three elongate strands 12. Other embodiments may have 1 or 2 elongate strands. The elongate strands 12 may be shaped, textured, or branched so that they are conducive to carrying cellular material. In still other embodiments, the cellular carrier 10 may feature more than 3 elongate strands 12. Embodiments with three or more elongate strands 12 may be advantageous because the strands naturally form an area between them that is suitable for carrying a cellular construct or other cellular material. Embodiments with three elongate strands 12 may be advantageous because they may be simpler to manufacture than embodiments with four or more strands. However, any number of strands may be used.

The thickness and length of the elongate strands 12 may vary. The suitable thickness and length may depend on the number of strands, the size of the insertion device, the desired shape of the strands, or the type of tissue into which the cellular carrier is being inserted. For example, it may be preferable to have relatively thin strands when a large number of strands are used. In contrast, one thick strand with a curved cross-sectional shape may be sufficient in other embodiments.

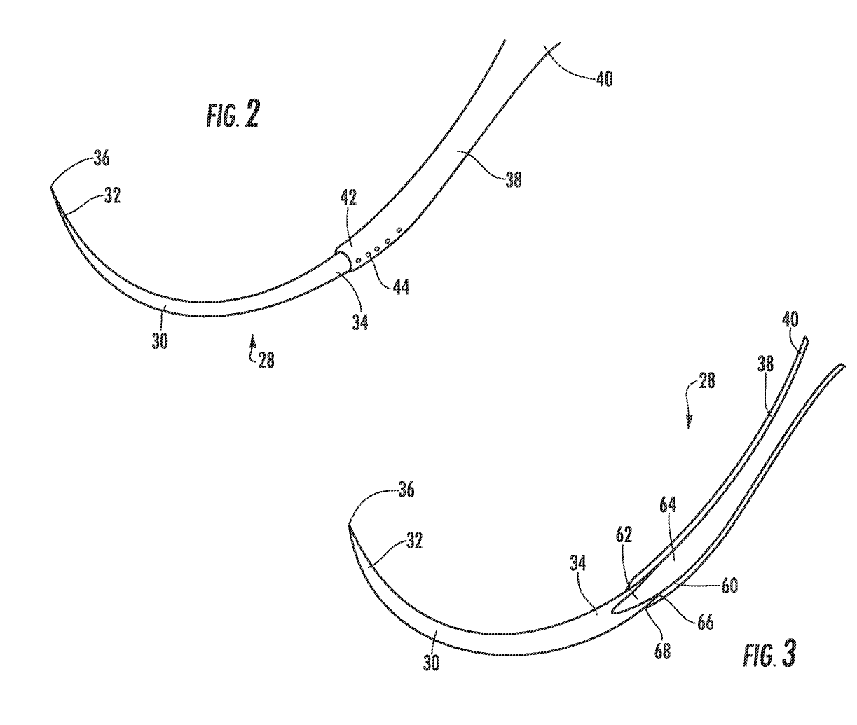

It is preferable that the length of the elongate strand 12 be approximately the length of a hollow tubular device, such as the hollow tubular body shown in FIGS. 2, 3, 4, and 5. However, embodiments with elongate strands 12 that have a length that is less than the length of the hollow tubular device may also be useful.

In embodiments with three or more elongate strands 12 like the one shown in FIG. 1, the elongate strands 12 may be melded together at their distal ends 14 to form a distal member 24. In these embodiments, the distal member 24 may be integral with the elongate strand 12 or strands 12. In other embodiments, the distal member 24 may be a separate piece that is connected to the elongate strand 12 or strands 12. The elongate strands 12 may also be melded together at their proximal ends 16 to form a stop member 18. In the alternative, the stop member 18 may be a separate piece that is connected to the elongate strands 12, i.e., the stop member may not be integrally formed with the proximal ends of the elongate strands. The stop member 18 has a distal end 20 and a proximal end 22.

The shape of the distal member and the stop member may vary. As shown in FIG. 1, the stop member 18 has a proximal end 22 that is narrower than the distal end 20. The distal end 20 of the stop member 18 is blunt, giving the stop member 18 a roughly triangular-shape. The blunt distal end 20 of the stop member 18 is shaped such that said distal end 20 catches against an exterior surface of tissue when the cellular carrier 10 is being moved through the tissue and thereby may cause the cellular carrier 10 to be embedded in the tissue, as explained further below with respect to FIGS. 10, 11, 12, and 13. These or any other shapes known in the art may be used for the stop member 18.

The distal member 24 may have a variety shapes. The distal member 24 may be connected to the distal end 14 of an elongate strand 12. It may also be integrally formed by melding more than one elongate strand 12 together. As shown in FIG. 1, the distal member 24 may be generally round. The distal member 24 may be triangular, circular, oval, amorphous, spade-shaped, elliptical, irregular, asymmetrical, symmetrical, or may be generally round, but with amorphous protrusions. These and any other shapes known in the art may be used for the distal member 24.

The stop member 18 may have a variety of shapes and configurations. The stop member may be parachute-like, round, oval, amorphous, spade-shaped, or have any other suitable shape. The stop member 18 may be directly connected to the elongate strands 12, as shown in FIG. 1. In the alternative, the stop member may be indirectly connected to the elongate strands 12 through the use of another piece of material.

The cellular carrier 10 is preferably made of polypropylene or a similar biocompatible material. The cellular carrier may also be made of any biocompatible material that is biodegradable or bioabsorbable, to allow the cellular carrier to be degraded or absorbed after it has been used to insert the cellular material. The cellular carrier 10 may also be composed of only one material or more than one material. The cellular carrier 10 may also be made of a radio-opaque material or comprise one or more radio-opaque components, to facilitate imaging of the carrier and construct. Likewise, the cellular construct may also be made of or comprise one or more radio-opaque components. The cellular carrier 10 and cellular construct may also be made of any other material known in the art. The cellular material may be coupled with the use of a scaffold material. For example, the cellular material may be carried on a scaffold of collagen fibrils.

The cellular material of the cellular construct preferably comprises cells. These cells are preferably stem cells capable of growing into the desired tissue for the operation being performed. The person of ordinary skill may also use other types of cellular material, such as a bioengineered molecule to attract a patient's cells and cause them to regenerate—rather than using cells from an external source. Any other cellular material known in the art may be used with the cellular carrier. In embodiments in which the cellular material comprises cells, the cellular construct is preferably grown on the cellular carrier. However, in some embodiments the cellular construct may be placed on the cellular carrier after cells have been grown.

It is desirable that the cellular carrier 10 be placed into an insertion device 28, such as the device shown in FIG. 2, for insertion into tissue. The insertion device 28 is formed of a needle 30 having a distal end 32 and a proximal end 34. The distal end 32 of the needle 30 has a sharpened distal tip 36. A hollow tubular body 38 with a proximal end 40 and a distal end 42 is attached to the needle 30.

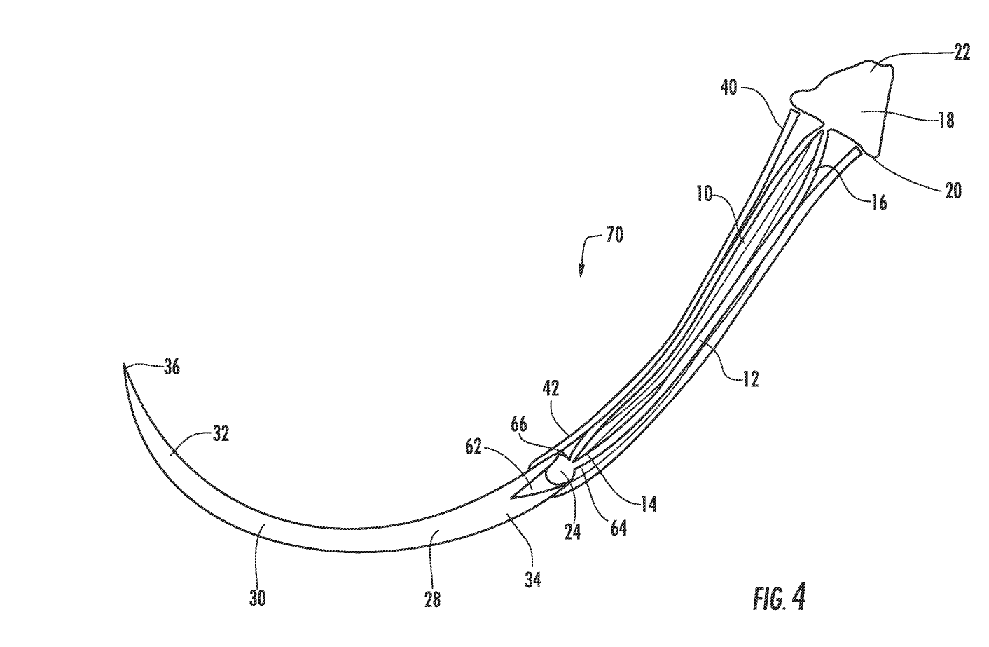

The connection between the hollow tubular body 38 and the needle 30 is accomplished by a connection between the proximal end 34 of the needle 30 and the distal end 42 of the hollow tubular body 38. FIGS. 3 and 4 show that the needle 30 may have a hollow proximal portion 62. The hollow proximal portion 62 of the needle 30 is connected to the hollow tubular body 38 such that together they form a single continuous hollow cavity 64. As shown in FIGS. 3 and 4, embodiments having a needle 30 with a hollow proximal portion 62 may have a most proximal portion 66 of the needle 30 that rests inside the diameter of the hollow tubular body 38. The most proximal portion 66 of the needle 30 may be slightly tapered as shown in FIGS. 3 and 4 to reduce the edges on the inside of the continuous hollow cavity 64. However, a needle with a blunt most proximal portion may also be used. As shown in FIG. 3, it is preferable that the most distal portion 68 of the hollow tubular body 38 overlaps with the most proximal portion 66 of the needle 30. Also, as shown in FIG. 3, it is preferable that the most distal portion 68 of the hollow tubular body 38 be tapered to make the exterior junction between the needle 30 and the hollow tubular body 38 as smooth as possible. This is advantageous because it may reduce the risk of the insertion device 28 catching on, becoming stuck in, or otherwise damaging the tissue.

FIG. 4 illustrates a cellular delivery device 70 formed of an insertion device 28 and a cellular carrier 10. The cellular carrier 10 has two elongate strands 12, each having a distal end 14 and a proximal end 16. The distal ends 14 of the elongate strands 12 are joined together to make a distal member 24. The distal member of FIG. 4 has a generally round shape. The proximal ends 16 of the elongate strands 12 are joined together to make a stop member 18, which has a proximal end 22 and a distal end 20. The insertion device 28 is composed of a needle 30 and a hollow tubular body 38, which are connected together. The needle 30 has a distal end 14, a proximal end 16, and a sharpened distal tip 36. FIG. 4 illustrates that the cellular carrier 10 may rest within the hollow tubular body 38 of the insertion device 28 such that the distal member 24 is located at least in part within the hollow proximal portion 62 of the needle 30. In other embodiments the distal member 24 may rest in the distal end 42 of the hollow tubular body 38 rather than in the hollow proximal portion 62 of the needle 30. It is preferable that the distal member 24 is sized so that the diameter of the distal member 24 is less than the inside diameter of the hollow tubular body 38 of the insertion device 28. This ensures that the distal member 24 will fit into the insertion device 28 and that the cellular carrier 10 will easily slide out of the hollow tubular body 38 of the insertion device 28 when desired.

FIG. 4 also shows a stop member 18 having a distal end 20 that is wider than the proximal end of the insertion device 28. This may be preferable because the stop member 18 will not easily follow the insertion device 28 through tissue.

In FIG. 4, the cellular carrier 10 is placed within the insertion device 28 such that the distal end 20 of the stop member 18 is nearly touching or is touching the proximal end 40 of the hollow tubular body 38. In other embodiments, the cellular carrier 10 may be positioned in the hollow tubular body 38 such that the stop member 18 is located some distance away from the proximal end 40 of the hollow tubular body 38.

In order to place the cellular carrier 10 within the hollow tubular body 38 as shown in FIG. 3, it is preferable that the cellular carrier 10 be guided into the proximal end 40 of the hollow tubular body 38. As shown in FIG. 4, the proximal end 40 of the hollow tubular body 38 may be flared to facilitate this process. This flaring creates an enlarged opening that may make it easier to guide the cellular carrier 10 into the proximal end 40 of the hollow tubular body 38.

Another feature that may aid in placing the cellular carrier 10 within the hollow tubular body 38 are one or more holes 44 near or at the distal end 42 of the hollow tubular body 38. The embodiment shown in FIG. 2 features five holes 44 at the distal end 42 of the hollow tubular body 38. In order to insert the cellular carrier 10 into the insertion device 28, the insertion device 28 and the cellular carrier 10 may be placed in a liquid medium. The cellular carrier 10 may be guided into the open proximal end 40 of the hollow tubular body 38. As the cellular carrier slides into the hollow tubular body 40 it may displace the liquid medium that is within the hollow tubular body 40. The holes 44 allow this displaced liquid medium to exit the insertion device 28 as the cellular carrier 10 is inserted. Other embodiments using this method may have more or fewer holes in the hollow tubular body than the embodiment shown in FIG. 2. In addition, the size, shape, placement, and configuration of the holes may vary. This method of insertion is exemplary only. Any other method mechanism for inserting a device into a cavity may be used.

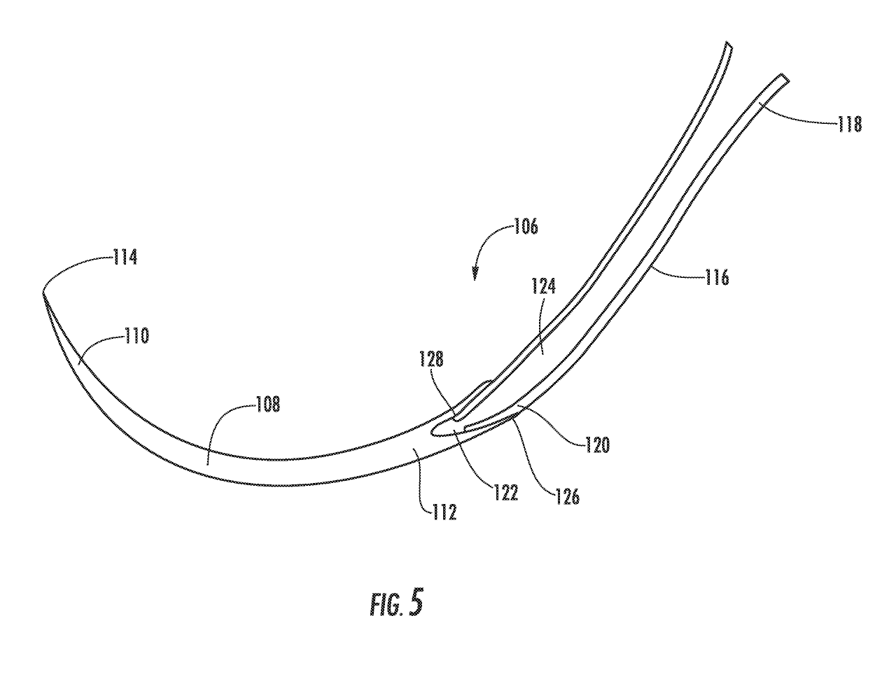

FIG. 5 illustrates another embodiment of an insertion device 106 composed of a needle 108 and a hollow tubular body 116. The needle 108 has a distal end 110 with a sharpened distal tip 114 and a proximal end 112. The hollow tubular body 116 has a proximal end 118 and a distal end 120. Like the embodiments shown in FIGS. 2, 3, and 4, the needle 108 has a hollow proximal portion 122 forming a continuous hollow cavity 124 in combination with the hollow tubular body 116. However, the connection between the needle 108 and the hollow tubular body 116 in FIG. 5 differs from the embodiments shown in FIGS. 2, 3, and 4. In FIG. 5, the most proximal portion 126 of the needle 108 is wider than the most distal portion 128 of the tubular body 116. Thus, the most distal portion 128 of the tubular body 116 rests within the hollow proximal portion 122 at the proximal end 112 of the needle 108. It is preferable in embodiments having this arrangement that the distal member of the cellular carrier is sized so that it cannot be placed distal to the most distal portion 128 of the hollow tubular body 116. This prevents the distal member from catching on the most distal portion 128 of the hollow tubular body 116. Is also preferable that the most distal portion 128 of the tubular body 116 and the most proximal portion 126 of the needle 108 be slightly tapered as shown in FIG. 5 to form the smoothest connection possible between the needle 108 and the hollow tubular body 116. However, other embodiments may not feature tapering.

The hollow tubular body and the needle shown in FIGS. 2, 3, 4, and 5 may be held together by frictional forces or frictional forces in combination with an adhesive. A notch, knob, or barb mechanism may also be used to securely connect the needle and hollow tubular body. Any other method for attaching or combination of methods for attaching known in the art may be used. It is preferable that the two components of the insertion device be securely connected together to ensure that they do not come apart during insertion into the tissue.

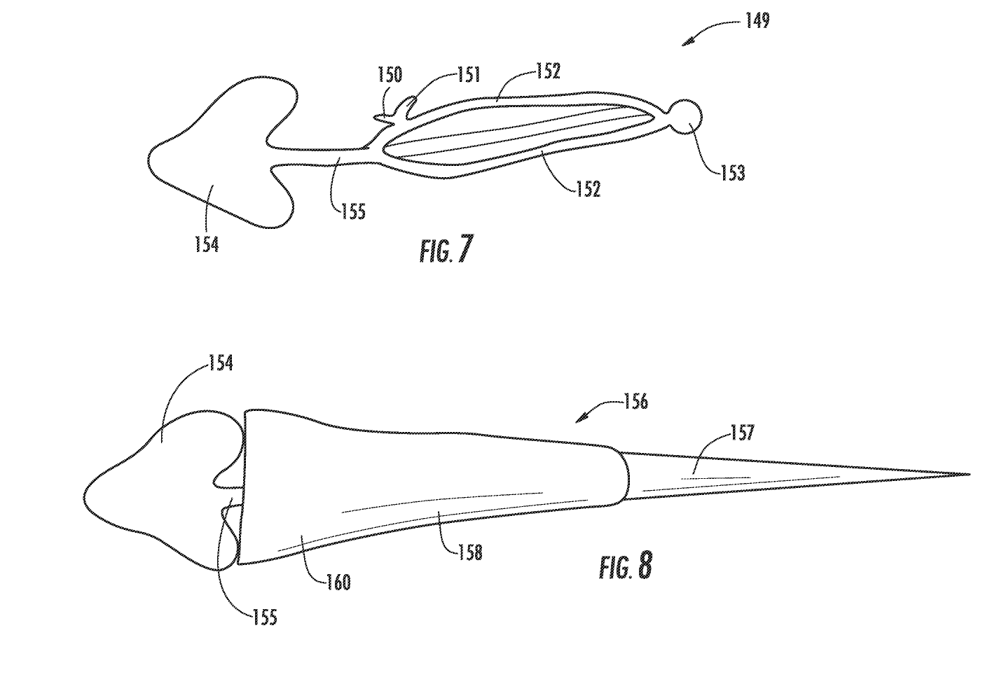

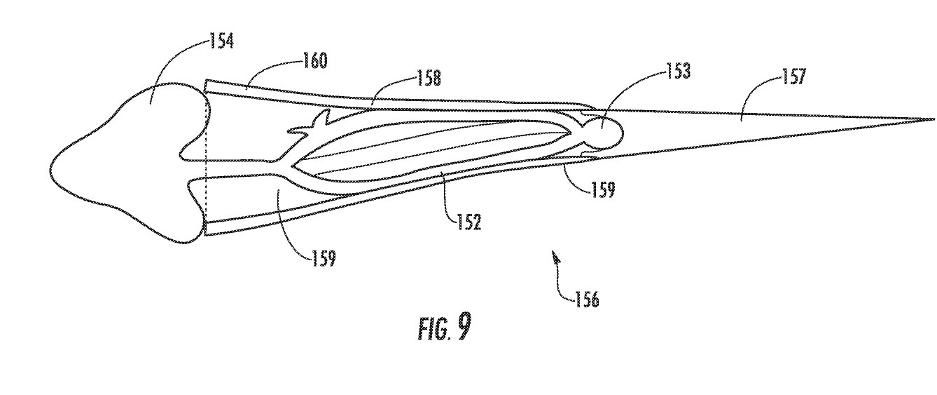

The size and shape of the needle used with the insertion device may vary. The size of the needle may vary depending on the medical procedure for which the needle is used. The shape of the needle may generally be curved or straight. The needle shown in FIGS. 2, 3, 4 and 5 is curved. The use of a curved needle may be advantageous because the shape allows a physician to easily insert the insertion device through a point on one surface of the tissue and then pass the insertion device out through a second point on the same surface of the tissue. The process of using an insertion device having a curved needle is described in greater detail with respect to FIGS. 10, 11, 12, and 13 below. The use of a straight needle is described in greater detail with respect to FIGS. 8 and 9 below.

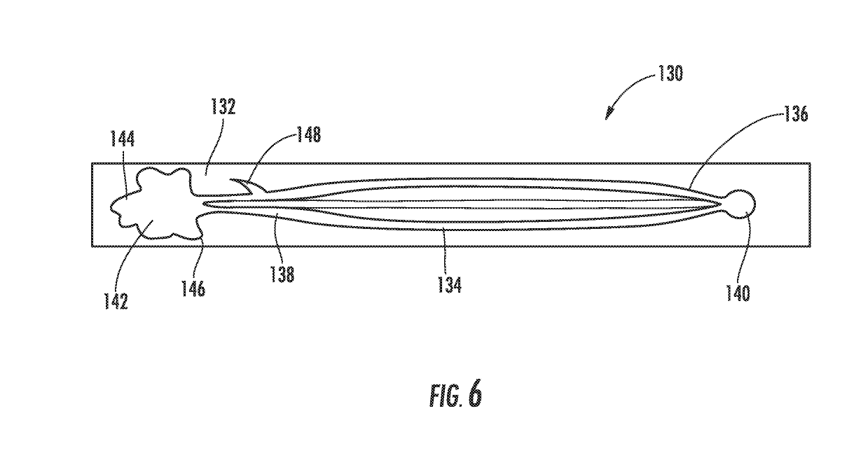

FIG. 6 shows an alternate embodiment of a cellular carrier 130. The alternate embodiment shown in FIG. 6 illustrates a distal member 140 having an oval shape. As shown in FIG. 6, the cellular carrier 130 may also feature at least one barb 148 on one or more elongate strand 134 or the distal member 140. FIG. 6 shows a barb 148 located on the proximal end 138 of an elongate strand 134. A barb 148 may also be located at the distal end 136 of one or more of the elongate strands 134 or along the center of one or more of the strands 134. In FIG. 6, the barb 148 is positioned such that it points toward the proximal end 138 of the elongate strand 134 and is on the exterior of the elongate strand 134—not within the space between the elongate strands 134. Thus, the barb 148 shown in FIG. 6 may aid in anchoring the cellular carrier 130 within the desired tissue by resisting movement in the proximal direction.

The composition, configuration, and number of barbs used on the cellular carrier may vary. Barbs may be configured to resist movement in the proximal direction as shown in FIG. 6, or in the distal direction. A barb positioned so that it points toward the distal end of the elongate strand 134 may resist movement in the distal direction. The barb 148 is preferably made of the same material as the elongate strand 134. However, the barb 148 may be made of any biocompatible, biodegradable, or bioabsorbable material known in the art. While FIG. 6 shows an embodiment with a single barb 148, other embodiments may have more than one barb or no barbs. Moreover, the width and length of the barb may vary depending on the type of tissue or procedure. The tip of the barb 148 is preferably not very sharp or stiff so as to avoid damaging the tissue of a patient. However, the sharpness and stiffness of the barb may vary.

FIG. 7 shows another alternate embodiment of a cellular carrier 149 having two barbs. The cellular carrier 149 shown in FIG. 7 features three elongate strands 152 which are connected to a round, ball-like distal member 153 at their distal ends. Along the length of one of the elongate strands 152 are two adjoining barbs, 150, 151. The distal barb 151 helps to prevent movement in the distal direction. In contrast, the proximal barb 150 may help to prevent undesired movement in the proximal direction. It may be useful to have barbs that prevent movement in either direction in order to ensure that the cellular carrier 149 is firmly planted in living tissue. In embodiments having multiple barbs, the barbs may be adjoining, as shown in FIGS. 7 and 8, or distanced from one another.

The alternate embodiment shown in FIG. 7 also features the use of a single solid strand of material called a tail 155. At the distal ends of the elongate strands 152, the elongate strands 152 are connected to the stop member 154 indirectly via a tail 155 formed of a solid piece of material. The tail thus forms the connection between the elongate strands 152 and the stop member 154.

FIGS. 8 and 9 illustrate an alternate embodiment of a cellular delivery device 156, which integrates the cellular carrier of FIG. 7. The cellular carrier 149 rests in the hollow cavity 159 of the cellular delivery device 156. In FIGS. 8 and 9, the cellular carrier 149 is slightly longer than the cavity 159 formed in the cellular delivery device 156. Thus, in FIG. 8, the stop member 154 of the cellular carrier 149 abuts the proximal end 160 of the hollow tubular body 158.

A tail 155, like the one shown in FIG. 7, may be advantageous because it may allow a user to implant cellular material at a targeted desired depth within living tissue without implanting the cellular material at shallower depths within the living tissue. In embodiments having a tail 155, the cellular material is carried on the elongate strands 152, but not on the tail 155. The tail 155 may be particularly useful for applications where the tissue in which the cellular material is being implanted has multiple layers. For example, the tissue may have a fatty surface layer that covers a layer of muscle. The user may wish to implant cellular material in the muscle, but not the fatty surface layer. When implanted in the living tissue, the cellular carrier will be positioned so that the stop member 154 abuts the outer surface of the tissue. The tail 155 will span the fatty layer and the elongate strands 152 carrying the cellular material will be in the deeper muscle layer. Thus, the tail 155 may allow the user to implant the cellular material in the muscle without implanting the material in the fatty surface layer. The use of a tail may serve a similar function in embodiments having 1, 2, or 4 or more elongate strands. Embodiments having a tail 155 may also feature one or more barbs on that tail.

The tail may vary in shape and size depending on the application. The length of the tail 155 may be varied according to the desired depth of implantation in the tissue. For applications in which the user seeks to implant the cellular material deep within the tissue, a longer tail 155 may be used. For applications in which the user seeks to implant the cellular material slightly below the surface of the tissue, a shorter tail 155 may be used. The width and shape of the tail may also vary depending on the application. The tail may have a variety of cross-sectional shapes. It may be generally round, oval, triangular, amorphous or have any other suitable shape. The tail may be integrally formed with one or more elongate strand or may comprise a separate piece that is attached to the elongate strand. The tail may also be integrally formed with the stop member. However, in other embodiments, the tail may comprise a separate piece that is attached to the stop member.

Unlike the embodiments shown in FIGS. 2, 3, 4, and 5, the alternate embodiment of a cellular delivery device shown in FIGS. 8 and 9 features a straight needle 157. A straight needle 157 may be passed through tissue in a manner similar to a curved needle. A straight needle 157 may be advantageous for applications in which the needle is pushed in one side of the living tissue and out the other side. It may also be advantageous to use a straight needle 157 when the device is being inserted into the body through the use of a catheter. Finally, physicians may be able to use a straight needle 157 in the same manner as a curved needle.

FIGS. 10, 11, 12, and 13 illustrate a method for delivering cellular material to living tissue in the human heart 162. The method involves the use of a cellular carrier 10 and an insertion device 28. The cellular carrier 10 of FIG. 1 and the insertion device of FIGS. 2, 3, and 4 is shown being used for this method in FIGS. 10, 11, 12, and 13. The cellular carrier 10 has three elongate strands 12 of material each with a distal end 14 and a proximal end 16. The cellular carrier 10 has a distal member 24 connected to the distal end 14 of the elongate strands 12, and a stop member 18 coupled to the proximal end 16 of the elongate strands 12. The insertion device 28 has a distal end 168 and a proximal end 170. The distal end 168 of the insertion device 28 is formed of a needle 30. The proximal end 170 of the insertion device 28 is formed of a hollow tubular body 38 and defines a cavity.

According to the method shown in FIGS. 10, 11, 12 and 13, cellular material 172 may be carried on the cellular carrier 10. The cellular material 172 may be placed or grown on the carrier 10. The cellular carrier 10 is received by the cavity in the proximal end 16 of the insertion device 28, such that the distal member 24 rests inside the cavity and the stop member 18 protrudes from the proximal end 170 of the insertion device 28. FIG. 10 shows the stop member 18 positioned so that the distal end of the stop member 18 abuts the proximal end 170 of the insertion device 28. In other embodiments, the stop member 18 may be located a distance proximal to the proximal end 170 of the insertion device 28. Together, the cellular carrier 10 and insertion device 28 form a cellular delivery device 70.

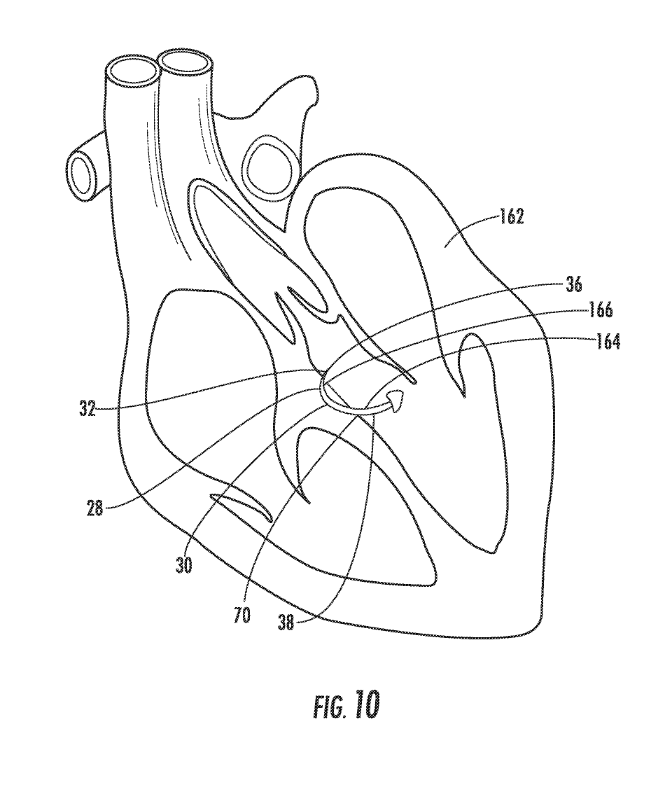

As shown in FIGS. 10 and 11, the cellular deliver device 70 is used to penetrate living tissue. A first point 164 in the surface of the living tissue is penetrated by the sharpened distal tip 36 of the needle 30 of the insertion device 28. The insertion device 28 is passed through the tissue and out of a second point 166 in the surface of the living tissue. The insertion device 164 naturally exits the tissue at a second point 166 due to the shape of the needle 30.

The insertion device 28 may be moved through the tissue by pushing the device though the first point 164. Once the sharpened distal tip 36 of the needle 30 penetrates the surface of the tissue at the second point 166, the needle 30 may be grasped at its distal end 32 and pulled until the proximal end 34 of the needle 30 exits at the second point 166. Any method for moving a needle through tissue known in the art may be used.

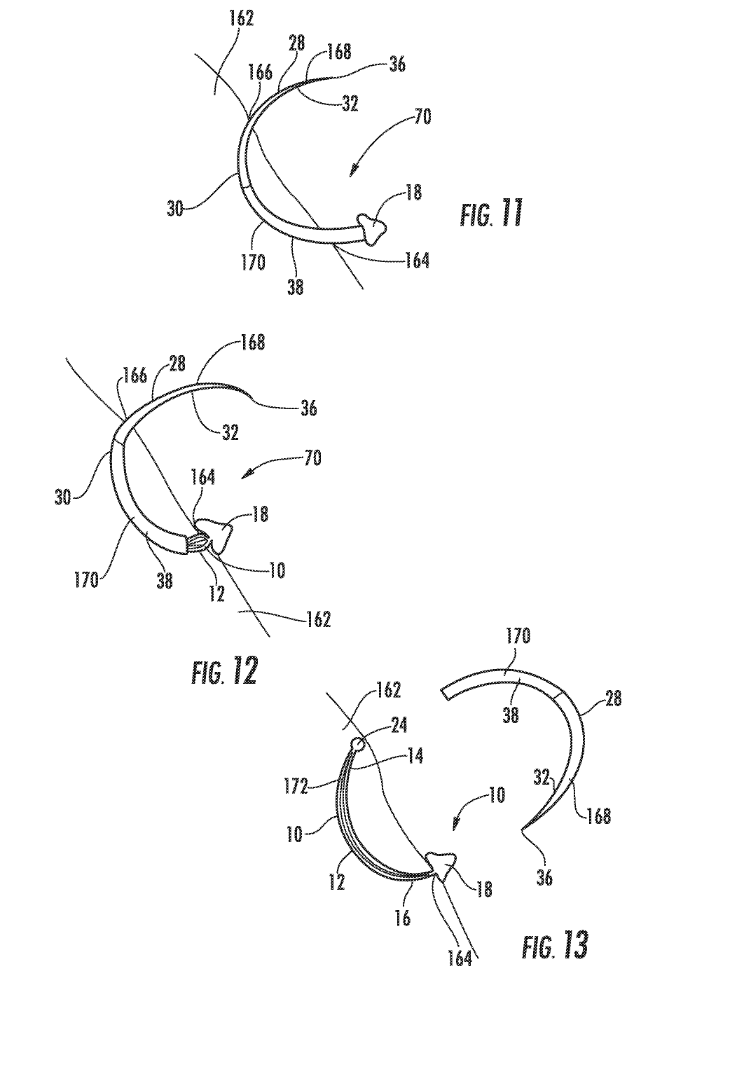

FIG. 12 illustrates that the stop member 142 catches on the surface of the tissue at the first point 164 in the surface of the tissue. This prevents the cellular carrier 10 from moving any further through the tissue. Thus, the insertion device 28 will continue to move through the tissue toward the second point 166, however, the cellular carrier 10 will remain in place within the tissue.

As shown in FIG. 13, the cellular carrier 10 is left embedded within the tissue after the insertion device 28 has been completely removed through the second point 166 in the surface of the tissue. Once in place, the patient's tissue may naturally shrink around the cellular carrier 10, holding it in place within the tissue. For added security, a barb 148 on one or more elongate strand 12 or on the distal member 24, such as the barb 148 shown in FIG. 6, may aid in holding the cellular carrier 10 in place. The barb 148 may be particularly useful in tissues that are not highly elastic. Once embedded in the tissue, the cellular material may take hold. In some embodiments, the cellular material on the cellular carrier 10 may be cells, which may grow and multiply once embedded.

As best seen in FIG. 4, it is preferable that the distal member 24 be sized in relation to the hollow tubular body 38 such that the outer diameter of the distal member 24 is slightly smaller than the inner diameter of the hollow tubular body 38. This size differential may be beneficial during the implantation of the cellular carrier 10 in the tissue as shown in FIGS. 10, 11, 12, and 13. It is advantageous for the distal member 24 to be smaller than the inner diameter of the hollow tubular body 38 because this allows the distal member 24 to move through the hollow tubular body 38 without getting stuck. The slight size differential between the outer diameter of the distal member and the inner diameter of the hollow tubular body is also advantageous because it allows the distal member to “clear” the hollow tubular body of any cellular material that may have become separated from the cellular carrier 10 as the cellular carrier 10 slides out of the cellular delivery device 70.

It is especially advantageous to have a distal member 24 with an outer diameter that is slightly smaller than the inner diameter of the hollow tubular body 38 in embodiments in which the cellular material is contained in a gelatinous cellular construct. Bits of gelatinous cellular construct may be prone to coming loose from the cellular carrier 10 as it slides out of the cellular delivery device 70 as shown in FIG. 12. Because the distal member 24 nearly fills the hollow tubular body 38, the distal member 24 works to catch any lose bits of gelatinous cellular construct and carries those bits out of the cellular delivery device 70. This ensures that all or nearly all of the cellular material on the construct becomes implanted in the tissue. The distal member 24 can also apply a compressive force to the cellular construct that is delivered by the distal member's connection to proximal end 18 through elongate strands 12. The compressive force counters any pulling force created by shearing forces from the inner surface of tubular body 38 that may break apart the cellular construct on delivery. Desirably, but not necessarily, the surface of the inner surface of tubular body 38 allows for smooth, non-adherent passage of cellular construct as it leaves cellular delivery device 70.

It may be useful to vary the length of the elongate strand 12 depending on the desired depth of insertion for the cellular material 172. The cellular material 172 is preferably located near the distal ends of the elongate strands 12. When shorter elongate strands 12 are used, the cellular material 172 may be embedded a short distance under the surface of the tissue. In contrast, longer elongate strands may be used to embed the cellular material 172 deeper below the surface of the tissue. In other embodiments, the cellular material 172 may be located along the majority or the entirety of the length of the elongate strands 12. In still other embodiments, the cellular material 172 may be placed near the centers or near the proximal ends of the elongate strands 12. In embodiments having a single elongate strand, the location of the cellular material 172 may be similarly varied.

As described, the foregoing cellular delivery device and method for using the cellular delivery device to deliver cellular material may be used to treat AV node failure. The device is particularly suited for treating AV node failure because it enables a cellular construct with cellular material to be delivered into the tissue rather than on top of the tissue. Within the tissue, the cellular material is less likely to be dislodged by the frequent contraction of the heart muscle. In embodiments in which the cellular material is cells, the cells are also more likely to get an adequate blood supply. The cells may be able to generate electrical signals and thus repair damage to the AV node—without the need for an external electrical stimulus. This method may also reduce the formation of scar tissue, which in turn, increases the likelihood that the cellular material will successfully take hold.

The device and method may also be used for any other application in which it is desirable to implant a construct or cellular material into tissue rather than on top of the tissue. For example, this device and method may be useful for linking together two ends of a nerve or the spinal cord. It may also be useful for linking muscles together in some types of reconstruction procedures, e.g., cases of severed limbs or digits. Depending on the application, any method known in the art for reaching the desired tissue may be used, e.g., open surgery or endovascular surgery.

While preferred embodiments of the invention have been described, it should be understood that the invention is not so limited, and modifications may be made without departing from the invention. The scope of the invention is defined by the appended claims, and all devices that come within the meaning of the claims, either literally or by equivalence, are intended to be embraced therein. Furthermore, the advantages described above are not necessarily the only advantages of the invention, and it is not necessarily expected that all of the described advantages will be achieved with every embodiment of the invention.

What is claimed is:

1. A device for delivering cellular material to tissue, said device comprising:
at least one elongate strand of biocompatible material having a distal end and a proximal end;
a stop member connected to said proximal end of said at least one elongate strand, said stop member having a proximal end and a distal end;
a distal member coupled to said distal end of said at least one elongate strand;
wherein said elongate strand forms a cellular carrier capable of carrying cellular material to living tissue and said stop member facilitates placement of said at least one elongate strand within said tissue.

2. The device of claim 1 wherein said device has at least three elongate strands, each elongate strand having a distal end and a proximal end, and said at least three elongate strands are melded together at said proximal ends to form said stop member, and said at least three elongate strands are melded together at said distal ends to form said distal member.

3. The cellular carrier device of claim 1 wherein said distal member comprises an oval shape.

4. The cellular carrier device of claim 1 wherein said cellular carrier comprises polypropylene.

5. The cellular carrier device of claim 1 wherein said at least one elongate strand further comprises a barb.

6. The device of claim 1 wherein said distal end of said stop member is configured to abut an outer surface of said tissue when said at least one elongate strand is disposed within said tissue.

7. A cellular delivery device, the device comprising:
a cellular carrier having at least one elongate strand of biocompatible material with a distal end and a proximal end, said cellular carrier further having a stop member connected to said proximal end of said at least one elongate strand and a distal member coupled to said distal end of said at least one elongate strand;
a needle having a distal end and a proximal end, said distal end of said needle having a sharpened distal tip;
a hollow tubular body having a proximal end and a distal end, said distal end of said tubular body being attached to said proximal end of said needle,
wherein said hollow tubular body is sized to receive said cellular carrier such that at least a portion of said at least one elongate strand is located within said hollow tubular body and said stop member protrudes from said proximal end of said tubular body.

8. The cellular delivery device of claim 7 wherein said needle is curved.

9. The cellular delivery device of claim 7 further comprising a single solid strand of biocompatible material connecting said stop member to said at least one elongate strand.

10. The cellular delivery device of claim 7 wherein said cellular carrier further comprises at least three elongate strands, each elongate strand having a distal end and a proximal end, and said at least three elongate strands are melded together at said proximal ends to form said stop member, and said at least three elongate strands are melded together at said distal ends to form said distal member.

11. The cellular delivery device of claim 7 wherein said cellular carrier device comprises polypropylene.

12. The cellular delivery device of claim 7 wherein said proximal end of said at least one elongate strand of said cellular carrier further comprises a barb.

13. The cellular delivery device of claim 7, wherein said needle has a hollow proximal end, wherein said hollow proximal end of said needle is connected to said hollow tubular body to form a continuous hollow cavity.

14. The cellular delivery device of claim 13, wherein at least a portion of said distal member of said cellular carrier rests in said hollow proximal end of said needle.

15. The cellular delivery device of claim 7, wherein said distal end of said hollow tubular body further comprises at least one hole to allow liquid to exit as said cellular carrier material is inserted into said proximal end of said tubular body.

16. The cellular delivery device of claim 7, wherein said proximal end of said tubular body is flared to facilitate insertion of said cellular carrier into said proximal end of said tubular body.

17. The cellular delivery device of claim 7, wherein said stop member has a distal end and a proximal end, said proximal end of said stop member being narrower than said distal end, said distal end being blunt such that said distal end catches against an exterior surface of tissue when said cellular carrier is being moved through said tissue and thereby causes said cellular carrier to be embedded in said tissue.

18. A method for delivering cellular material to living tissue, comprising the steps of:
providing a cellular carrier having at least one elongate strand of material with a distal end and a proximal end, said cellular carrier having a distal member coupled to said distal end of said at least one elongate strand, and a stop member connected to said proximal end of said at least one elongate strand;
providing an insertion device with a distal end formed of a needle and a proximal end formed of a tubular body, said proximal end of said insertion device defining a cavity;
carrying cellular material on said cellular carrier;
receiving said cellular carrier in said cavity in said proximal end of said insertion device, such that said distal member rests inside said cavity and said stop member protrudes from said proximal end of said insertion device;
penetrating a first point in a surface of living tissue with said needle of said insertion device;
passing said insertion device through said tissue and out of a second point in said surface of said living tissue; and
allowing said stop member to catch on said surface of living tissue, thereby preventing said cellular carrier from moving any further through said tissue so that said cellular carrier is left embedded within said tissue after said insertion device is removed.

19. The method of claim 18 further comprising the step of growing cells on said cellular carrier.

20. The method of claim 19 further comprising growing said cells on said cellular carrier by immersing said cellular carrier in a liquid medium.
